(19) United States
(12) Patent Application Publication
(10) Pub. No.: US 2026/0091225 A1
(43) Pub. Date: Apr. 2, 2026

(54) TRANSDUCER ARRAY WITH ELECTRICALLY CONNECTED SUBASSEMBLIES

(71) Applicant: Novocure GmbH, Baar (CH)

(72) Inventors: Noa HALAVEE, Tel Aviv (IL); Elie YAACOBI, Haifa (IL); Dmitry GOLOM, Haifa (IL)

(73) Assignee: Novocure GmbH, Baar (CH)

(21) Appl. No.: 19/343,687

(22) Filed: Sep. 29, 2025

Related U.S. Application Data
(60) Provisional application No. 63/701,217, filed on Sep. 30, 2024.

(51) Int. Cl. A61N 1/36; A61N 1/04

(52) U.S. Cl. CPC A61N 1/36002; A61N 1/0476; A61N 1/0496

(57) ABSTRACT

A transducer apparatus for delivering tumor treating fields to a subject's body, the transducer apparatus including: first and second electrode subassemblies, each subassembly configured to be positioned over the subject's body with a front face of the subassembly facing the subject's body, each subassembly having a back face opposite the front face, each subassembly comprising: at least one electrode element; an anisotropic material layer electrically coupled to the at least one electrode element, the anisotropic material layer comprising a front face facing the subject's body and a back face opposite the front face, the back face facing the at least one first electrode element; and a flexible electrical connector electrically connecting the first and second electrode subassemblies, wherein the first and second electrode subassemblies are separated by the flexible electrical connector.

### CROSS-REFERENCE TO RELATED APPLICATIONS

This application claims priority to U.S. Provisional Patent Application No. 63/701,217, filed Sep. 30, 2024, which is incorporated herein by reference in its entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into a region of interest by electrode assemblies (also known as “transducer arrays” or “transducers”) placed on the subject's body and applying alternating current (AC) voltages between the transducers. Conventionally, transducers used to generate TTFields include a plurality of electrode elements comprising ceramic disks. One side of each ceramic disk is positioned against the subject's skin, and the other side of each disc has a conductive backing. Electrical signals are applied to this conductive backing, and these signals are capacitively coupled into the subject's body through the ceramic discs. Conventional transducer designs include rectangular arrays of ceramic disks aligned with each other in straight rows and columns and attached to the subject's body via adhesive.

### BRIEF DESCRIPTION OF THE DRAWINGS

FIGS. 1A and 1B depict a top plan view (i.e., a back face plan view) and a bottom plan view (i.e., a front face view), respectively, of an example transducer apparatus according to some embodiments.

FIG. 1C depicts a cross-section view of the example transducer apparatus of FIGS. 1A and 1B taken across section 1C-1C′ according to some embodiments.

FIG. 1D depicts a cross-section view of the example transducer apparatus of FIGS. 1A and 1B taken across section 1D-1D′ according to some embodiments.

FIGS. 1E, 1F, and 1G depict cross-section views of example transducer apparatuses according to some embodiments.

FIGS. 2A to 2D depict top plan views (i.e., back face plan views) of example transducer apparatuses according to some embodiments.

FIGS. 3A to 3D depict perspective views of example transducer apparatuses on a mannequin according to some embodiments.

FIG. 4 is a flowchart depicting an example method of applying TTFields to a subject's body according to some embodiments.

Various embodiments are described in detail below with reference to the accompanying drawings, wherein like reference numerals represent like elements, and wherein descriptions of like elements may not be repeated for every embodiment, but may be considered to be the same if previously described herein.

The figures provided herein are for illustrative purposes and may not be to scale. Variations in dimensions, proportions, and configurations may exist between the figures and the actual embodiments. The figures are intended to facilitate understanding of the embodiments and should not be construed as limiting the scope of the disclosure.

### DESCRIPTION OF EMBODIMENTS

This application describes exemplary transducer apparatuses used to apply TTFields to a subject's body, for example, for treating one or more cancers. This application also describes exemplary methods to apply TTFields to a subject's body using transducers.

Transducers used to apply TTFields to a subject's body often include multiple electrode elements electrically coupled together on a substrate and attached to the subject's body at a desired location, for example, via an adhesive backing of the substrate or a separately applied adhesive. Proper positioning of the transducer apparatus over a target region (e.g., a tumor) can affect performance of treatment. However, proper placement can be difficult, depending on where the transducer apparatus is being placed and particularly when the subject is placing the transducer apparatus without assistance or even with the assistance of a caregiver. Improper positioning may lead to diminished effectiveness. Further, positioning of the transducer apparatus over certain placement areas (e.g. on a subject's sternum, back, and oblique/latissimus dorsi area) are difficult due to the contour of the areas and bi-direction tension that results in a risk of detachment of the transducer apparatus when the subject shifts or moves. Positioning over certain placement areas (e.g. a subject's sternum) is also difficult due to sweating of the subject, which may also result in a risk of detachment. Accordingly, difficulties in positioning may lead to a decrease in the performance of the treatment. Accordingly, a way to assist with properly positioning one or more electrode arrays is desirable.

The inventors have discovered that utilizing two or more electrode subassemblies coupled together with a flexible electrical coupling may facilitate and enable the positioning of the transducer apparatus in the proper (e.g., optimized) position and may improve the electrical connection of the transducer apparatus to the subject. The flexible electrical coupling may also bend such that it forms a substantially concave shape away from the subject's body, thereby allowing improved heat dissipation.

Further, as recognized by the inventors, the flexible electrical coupling may come into contact with the subject's skin and may cause irritation. Due to a desire by the subject to reduce any discomfort caused by the transducer apparatus, the subject may decrease using the transducer apparatus, which may result in the subject receiving less than a desired dosage of TTFields. The inventors have discovered that a foam layer coupled to the flexible electrical coupling (e.g., on the side facing the subject's body) may help alleviate this discomfort.

Additionally, treatment often requires using one or more pairs of transducer apparatuses. Conventionally, each transducer apparatus may require its own cable, which can be undesirable for many reasons. For example, multiple cables can lead to complications from discomfort due to tangling of cables and/or, in some situations, pulling on or detachment of one or more cables from the transducer apparatuses. The inventors have discovered that utilizing two or more electrode subassemblies coupled together with a flexible coupling further reduces the amount of cables required.

Furthermore, conventional transducers have large, rectangular surfaces for applying TTFields to the subject's body. However, subjects can experience skin irritation on portions of their skin that are contacted by the transducers during TTFields treatment. Such irritation may be common at positions directly underneath the electrode elements, where heat and current may be at their highest concentrations.

As recognized by the inventors, on transducer arrays that comprise multiple electrode elements, the portions of the transducer arrays positioned directly beneath the electrode elements may become hotter than the portions of the transducer arrays positioned between the electrode elements. Furthermore, higher currents flow through the electrode elements that may be located along the edge of the array compared to the electrode elements located toward the middle of the array. Further still, an electrode element located at a corner or similar sharp bend in the edge of the array may have a higher current than other electrode elements along the edge and near the center of the array.

As recognized by the inventors, an uneven distribution of current through the transducer array may lead to higher temperature zones (or “hot spots”), e.g., at the corners or edges of the transducer array, which, in turn, may limit the maximum operational current that may be driven by a transducer array and, as a result, the strength of the resulting TTFields.

As recognized by the inventors, a transducer apparatus may also include an anisotropic material layer located on a side of the array of electrode elements facing the subject's body. Such an anisotropic material layer may spread the heat and/or current generated at the individual electrode elements within a plane that is perpendicular to the direction from the electrode elements to the subject's body. Spreading heat and/or current in this plane may reduce the concentration of heat and/or current at locations directly under the individual electrode elements, thus reducing the amount or severity of irritation, if any, that occurs on the subject's skin.

As further recognized by the inventors, a transducer apparatus including an anisotropic material layer may cause irritation to the subject due to the sharp edges of the anisotropic material layer. When a piece of anisotropic material is cut to size to be used as an anisotropic material layer in a transducer apparatus, the edges of the transducer apparatus may be sharp. As such, when these edges come in contact with a subject, the subject may experience discomfort. Due to a desire by the subject to reduce this discomfort from the transducer apparatus, the transducer apparatus may be placed in a different non-desired position on the subject for delivery of TTFields, which may result in the subject receiving less than a desired dosage of TTFields. Further, due to a desire by the subject to reduce this discomfort from the transducer apparatus, the subject may decrease the period(s) of time spent using the transducer apparatus, which may result in the subject receiving less than a desired dosage of TTFields. The inventors have discovered that using a foam layer with the anisotropic material layer (for example, in locations on the skin-facing side (i.e., the front face, or the bottom) of the anisotropic material layer) may help to alleviate this discomfort experienced by a subject. The inventors have further discovered that the foam layer may advantageously provide some additional flexibility for the array when the subject moves or twists the body part associated with the location of the array.

Descriptions of embodiments related to specific exemplary drawings herein may be applicable, and may be combined with, descriptions of embodiments related to other exemplary drawings herein unless otherwise indicated herein or otherwise clearly contradicted by context.

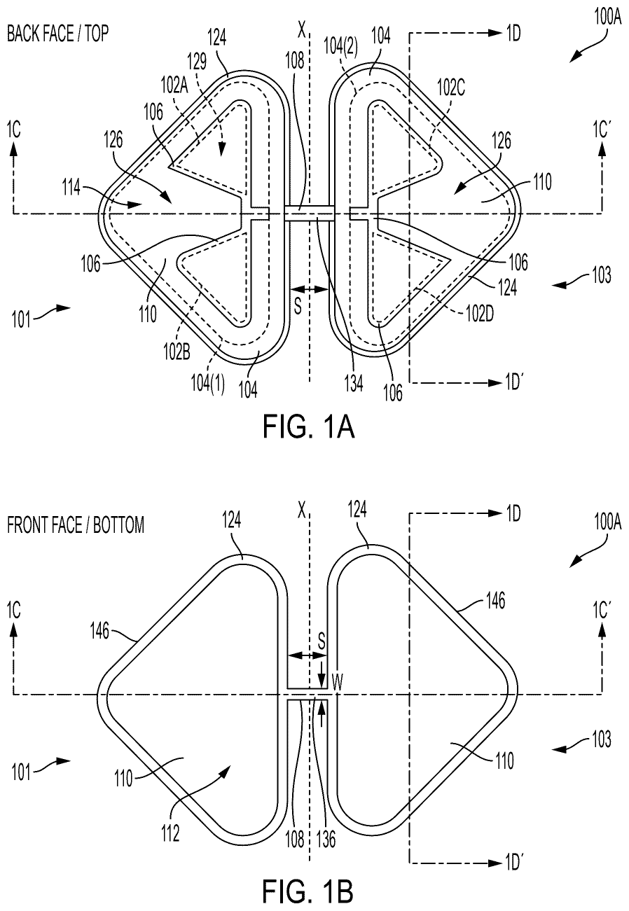

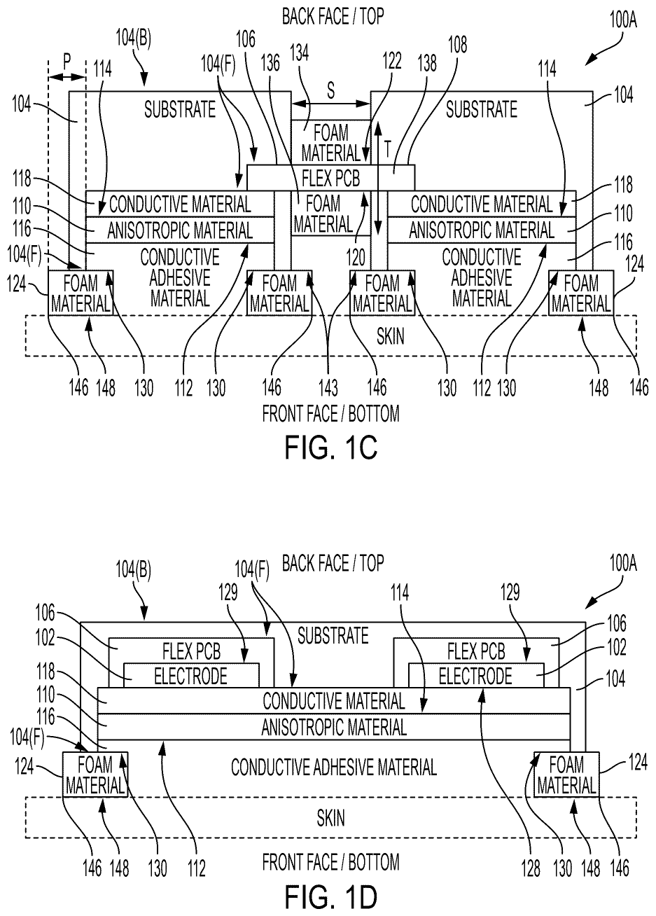

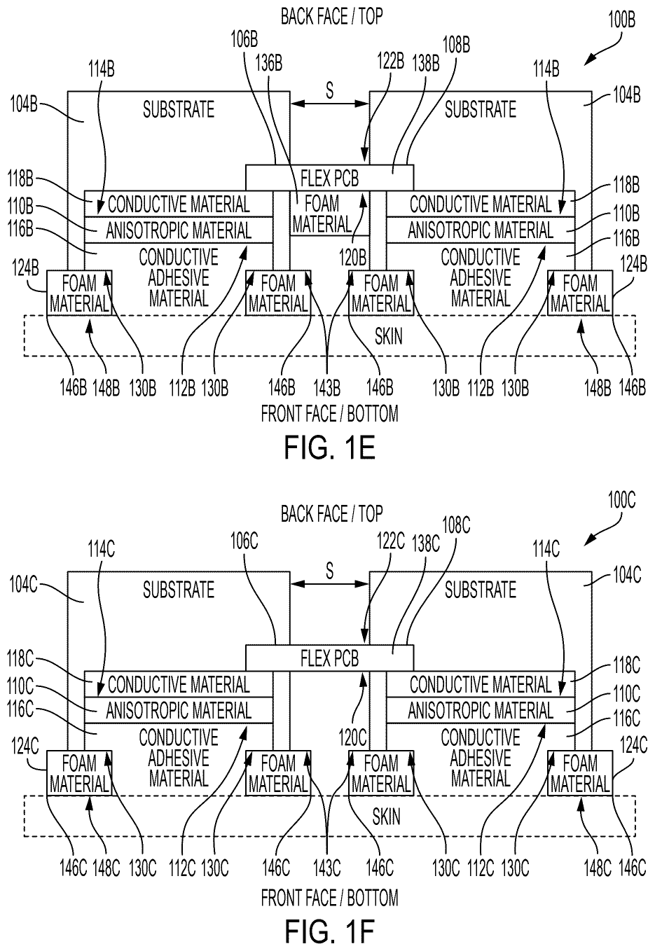

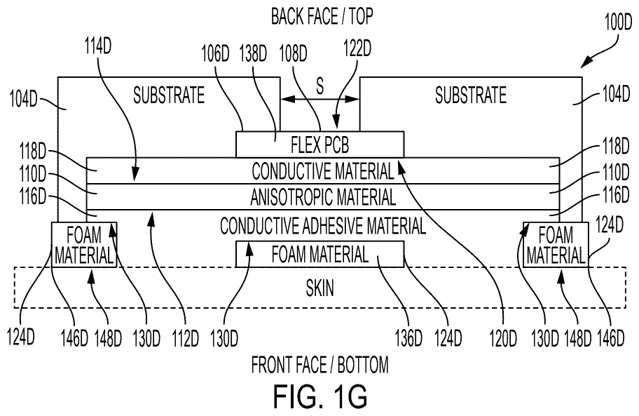

FIGS. 1A and 1B depict a top plan view (i.e., a back face plan view) and a bottom view (i.e., a front face view, or a skin-facing view), respectively, of an example transducer apparatus 100A according to some embodiments. FIG. 1C depicts a cross-section view of the example transducer apparatus 100A of FIGS. 1A and 1B taken across section 1C-1C′ according to some embodiments, and FIG. 1D depicts a cross-section view of the example transducer apparatus 100A of FIGS. 1A and 1B taken across section 1D-1D′ according to some embodiments. FIGS. 1A and 1B illustrate the transducer apparatus 100A as viewed from a direction perpendicular to a face of the transducer apparatus 100A. FIG. 1A illustrates a plan view from the back side of the transducer apparatus 100A (i.e., the side facing away from the subject's body, or the back face, or the top). FIG. 1B illustrates the front side of the transducer apparatus 100A (i.e., the side facing the subject's body, or the front face, or the bottom).

As shown in FIGS. 1A and 1B, the transducer apparatus 100A may include a first electrode subassembly (or first transducer subassembly) 101 and a second electrode subassembly (or second transducer subassembly) 103. The first electrode subassembly 101 and the second electrode subassembly 103 may have shapes that are symmetrical along the sagittal plane X and include the same components. Thus the following description of the components apply to both the first and the second electrode subassemblies 101, 103.

Each of the first and the second electrode subassemblies 101, 103 may include at least one electrode element 102 (i.e., 102A, 102B, 102C, 102D), a flexible printed circuit board (PCB) 106 electrically connecting the electrode elements 102, an anisotropic material layer 110 electrically coupled to the at least one electrode element 102, and a substrate 104 for holding the at least one electrode element and the anisotropic material layer against the subject's body. The first and the second electrode subassemblies 101, 103 may be connected with a flexible electrical connector 108, where the flexible electrical connector 108 separates the first and the second electrode subassemblies 101, 103. In FIG. 1A, the electrode elements 102A, 102B, 102C, and 102D are shown in dashed outline, as they are located between the flexible PCB 106 and the anisotropic material layer 110. In some embodiments, electrode elements 102 may be attached to or positioned on the flexible PCB 106. In other embodiments, the electrode elements 102 may be a component of or positioned within the flexible PCB 106. In FIG. 1A, although the substrate 104 covers the back sides of the first and the second electrode subassemblies 101, 103, the substrate 104 is shown as a cut-away in partial view by the dashed lines 104(1) and 104(2) so as to illustrate the components beneath the substrate 104. As illustrated in FIGS. 1C and 1D, the substrate 104 covers the back sides of first and second electrode subassemblies 101, 103.

When viewed in a direction perpendicular to and toward the back face of the subassemblies 101, 103 (as in FIG. 1A), the first and second electrode subassemblies 101, 103 may have a same shape (or mirror image thereof). In some embodiments, each of the first and second electrode subassemblies 101, 103 may be triangular shaped, substantially triangular shaped, rounded triangular shaped, substantially rounded triangular shaped, kidney bean or jelly bean shaped, substantially kidney bean or jelly bean shaped, ovoid shaped, substantially ovoid shaped, oval shaped, or substantially oval shaped, or each subassembly has an asymmetric oval, ovaloid, ovoid, or ovate shape or a stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature. For example, in FIG. 1A, the first and second electrode subassemblies 101, 103 may be triangular or rounded triangular shaped.

In some embodiments, the first and the second electrode subassemblies 101, 103 may be the same size, have the same shape, have the same dimensions, and have the same surface area. In some embodiments, the first and the second electrode subassemblies 101, 103 may be different sizes and/or shapes. For example, one of the electrode subassemblies may have a surface area at least 25% and at most 50% larger (including any percentage therebetween) than the surface area of the other electrode subassembly.

Each of the first and the second electrode subassemblies 101, 103 may include at least one electrode element 102. In some embodiments, each of the first and the second electrode subassemblies 101, 103 has at least two electrode elements 102. In some embodiments, each of the first and the second electrode subassemblies 101, 103 has only one electrode element 102. In some embodiments, the subassemblies 101, 103 have the same number of electrode elements 102. In some embodiments, the subassemblies 101, 103 have a different number of electrode elements 102.

The first and the second electrode subassemblies 101, 103 may include the same number or different numbers of electrodes elements 102. In some embodiments, the first and second electrode subassemblies 101, 103 may include one, two, three, four, five, six, seven, eight, nine, ten, eleven, twelve, thirteen, fourteen, fifteen, sixteen, seventeen, eighteen, nineteen, twenty, or more electrodes elements 102. For example, in FIG. 1A, the first and the second electrode subassemblies 101, 103 are depicted as having two electrodes elements 102 each. The first electrode subassembly 101 includes two electrodes 102A, 102B, and the second electrode subassembly 103 includes two electrodes 102C, 102D.

In some embodiments, the electrodes elements 102 may have the same shape. In some embodiments, when viewed in a direction perpendicular to and toward the back face of the subassemblies, the electrodes element 102 may be triangular shaped, substantially triangular shaped, rounded triangular shaped, substantially rounded triangular shaped, kidney bean or jelly bean shaped, substantially kidney bean or jelly bean shaped, ovoid shaped, substantially ovoid shaped, oval shaped, or substantially oval shaped, or each subassembly has an asymmetric oval, ovaloid, ovoid, or ovate shape or a stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature. For example, in FIG. 1A, the first and the second electrode subassemblies 101, 103 are depicted as having two triangular shaped (or rounded triangular shaped) electrodes elements each. The first electrode subassembly 101 includes two triangular or rounded triangular shaped electrodes 102A, 102B, and the second electrode subassembly 103 includes two triangular or rounded triangular shaped electrodes 102C, 102D.

The electrodes elements 102 of the first and the second electrode subassemblies 101, 103 may be adapted to administer TTFields therapy to a subject. The electrodes elements 102 may be electrically conductive and may be substantially flat. The electrode elements 102 may or may not be capacitively coupled. The electrodes elements 102 may be metal, metal alloy, layered metal, laminated conductive material, or may be ceramic or non-ceramic dielectric material positioned over a flat conductor (e.g., polymer film disposed on flat metal). In some embodiments, the electrode elements do not have a dielectric material.

In some embodiments, the dielectric material of the electrode elements 102 may have a dielectric constant ranging from 10 to 50,000. In some embodiments, the layer of dielectric material comprises a high dielectric polymer material such as poly(vinylidene fluoride-trifluoroethylene-chlorotrifluoroethylene) and/or poly(vinylidene fluoride-trifluoroethylene-1-chlorofluoroethylene). Those two polymers are abbreviated herein as “Poly(VDF-TrFE-CTFE)” and “Poly(VDF-TrFE-CFE),” respectively. The dielectric constant of these materials may be on the order of 40. In some embodiments, the polymer layer may be poly(vinylidene fluoride-trifluoroethylene-chlorotrifluoroethylene-chlorofluoroethylene) or “Poly(VDF-TrFE-CTFE-CFE).”

In some embodiments, the layer of dielectric material of the electrode elements 102 comprises a terpolymer comprising polymerized units of monomers such as VDF, TrFE, CFE and/or CTFE in any suitable molar ratio. Suitable terpolymers include those, for example, having 30 to 80 mol % VDF, 5 to 60 mol % TrFE, with CFE and/or CTFE constituting the balance of the mol % of the terpolymer.

The flexible PCB 106 may electrically couple together the electrode elements 102. In some embodiments, the flexible PCB 106 may cover the back faces 129 (FIGS. 1A, 1D) of the electrode elements 102. An edge of the electrode element 102 may be offset inwardly from an edge of the flexible PCB 106. In some embodiments, when viewed in a direction perpendicular to and toward the back face of the electrode subassemblies 101, 103, the flexible PCB 106 may cover up to and including 15%, 20%, 30%, 40%, 60%, 80%, or any percentage therebetween of a back face 114 (FIGS. 1A, 1C, 1D) of the anisotropic material layer 110. The flexible PCB 106 does not function as an electrode. The flexible PCB 106 may be a non-adhesive region. In some embodiments, the flexible PCB 106 may comprise a polyimide film, such as, for example, Kapton® (from DuPont, Wilmington, DE, USA) and conductive traces (for example, and without limitation, copper traces or traces of conductive ink, etc.).

In some embodiments, each subassembly 101, 103, may have one or more temperature sensors (such as, for example thermistors) (not shown) associated with some or all electrode elements 102 of the at least one electrode element 102 of each subassembly 101, 103.

The flexible electrical connector 108 may extend between and electrically couple the first and the second electrode subassemblies 101, 103. In some embodiments, the flexible electrical connector 108 may include a portion 138 of the flexible PCB 106 to electrically connect the first and the second electrode subassemblies 101, 103 (FIG. 1C). The portion 138 of the flexible PCB 106 may be part of neither the first nor the second electrode subassemblies 101, 103. The flexible electrical connector 108 may electrically connect the at least one electrode element 102 of the first electrode subassembly 101 and the at least one electrode element 102 of the second electrode subassembly 103.

The flexible electrical connector 108 may include a front face 120 facing the subject's body and a back face 122 opposite the front face. The front face 120 of the flexible electrical connector 108 may be free of adhesive material (FIG. 1C). When the subassemblies 101, 103 are viewed in cross-section, the flexible electrical connector 108 may be adapted not to be adhesively held to the subject's body.

The flexible electrical connector 108 may optionally comprise a conformal coating that covers or wraps around it, such as, for example, a polyethylene, polypropylene or polyethylene terephthalate coating (or any other polymer), which may be shrink-wrapped around the flexible electrical connector 108. Alternatively, or in addition, the flexible electrical connector 108 may further include a foam material layer 134 on the back face 122 of the flexible electrical connector 108 and/or a foam material layer 136 on the front face 120 of the flexible electrical connector 108. In some embodiments, the flexible electrical connector 108 may be covered on all sides by a foam layer. In some embodiments, the flexible electrical connector 108 may be a layered component including the foam material layer 134, the portion 138 of the flexible PCB 106, and the foam material layer 136. In some embodiments, the flexible electrical connector 108 may be or may include a plastic coated wire to electrically connect the first and the second electrode subassemblies 101, 103. In some embodiments, one or more wedges may reside between the flexible electrical connector 108 and the subject's body, and the one or more wedges may comprise foam material.

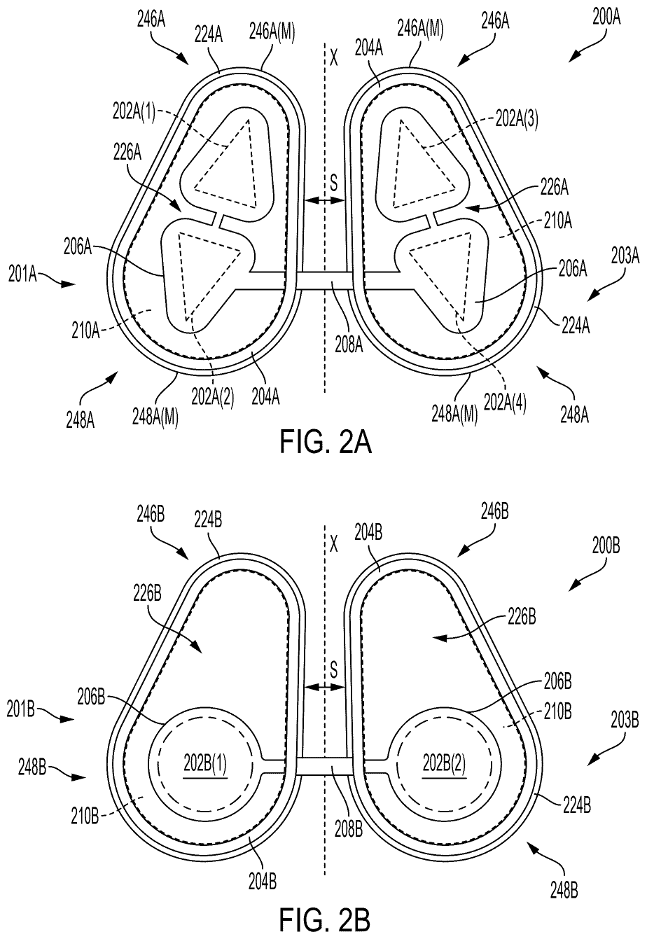

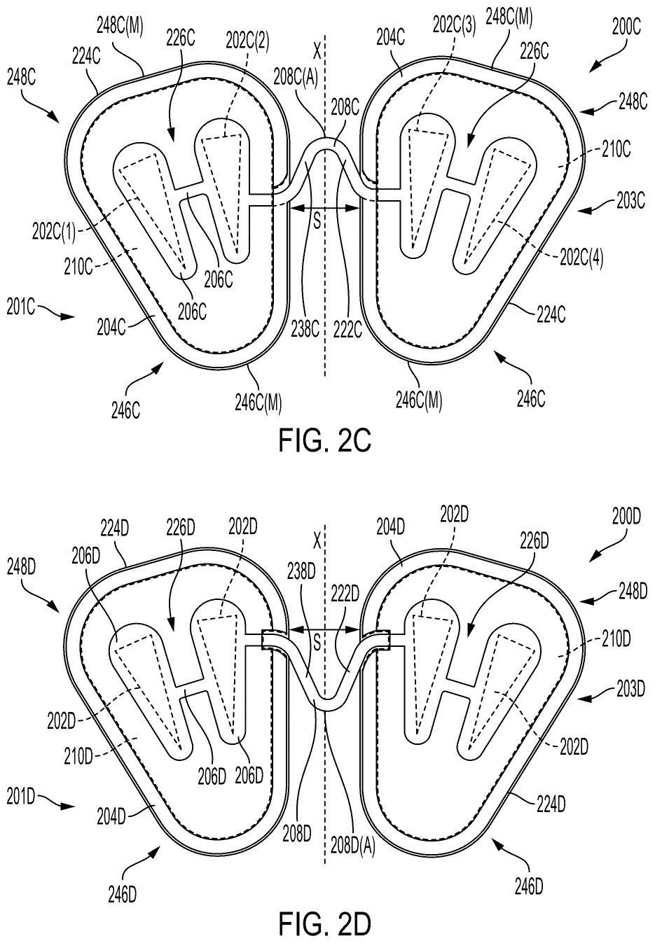

In some embodiments, when viewed in a direction perpendicular to and toward the back face of the subassemblies 101, 103 (as in FIG. 1A), the flexible electrical connector 108 may have a substantially linear shape (e.g., FIGS. 1A, 2A, 2B). In some embodiments, when viewed in a direction perpendicular to and toward the back face of the subassemblies 101, 103, the flexible electrical connector 108 may have different shape, such as, for example: a U-shape, substantially U-shape, or rounded V-shape (e.g., FIGS. 2C, 2D); a shape with two or more bends, such as an undulating shape, serpentine shape, wavelike shape, zig-zag shape; a shape with a combination of concave and convex portions; or a spiral shape.

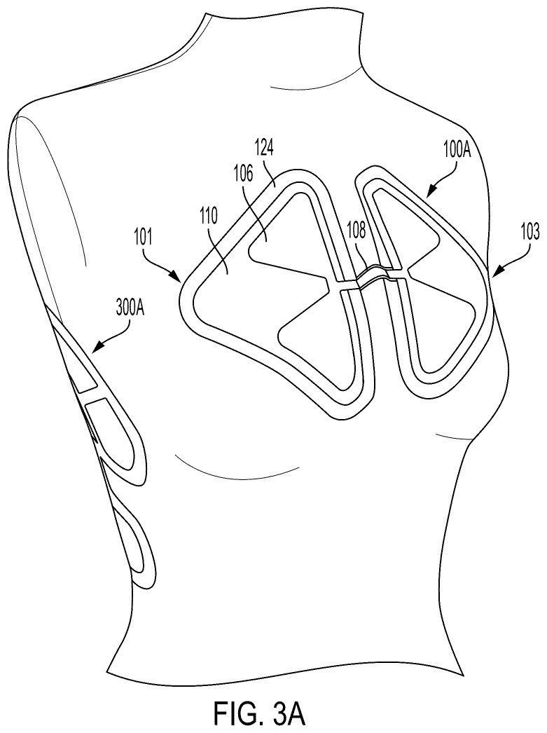

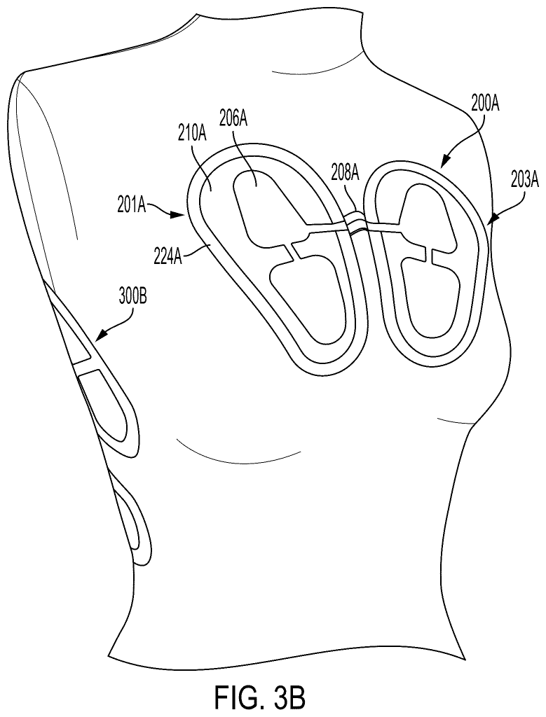

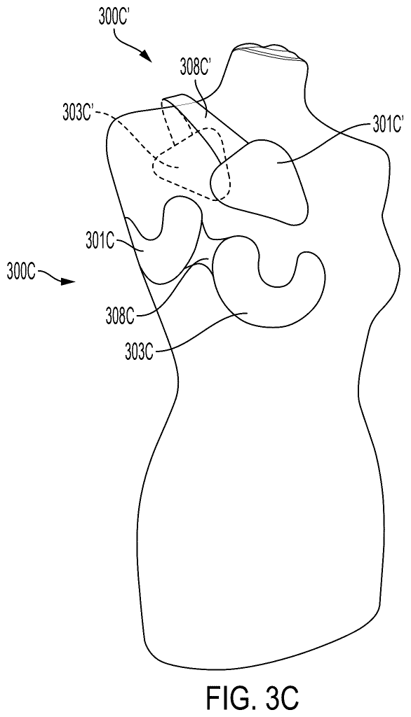

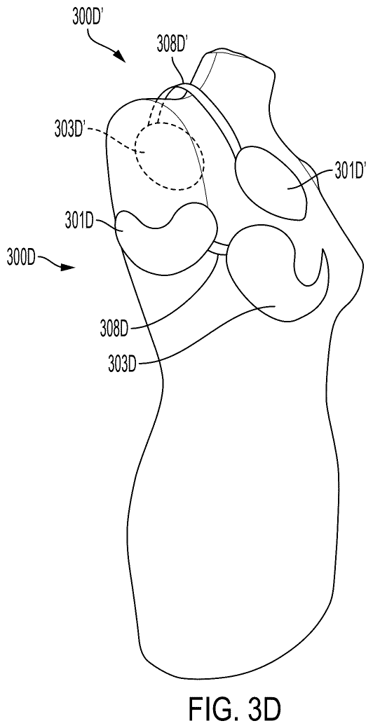

In some embodiments, the flexible electrical connector 108 may be adapted to be deformed to a substantially concave shape away from the subject's body (e.g., FIGS. 3A, 3B). In some embodiments, when the subassemblies are viewed in cross-section, the flexible electrical connector may be adapted to deform from a substantially planar shape to a U-shape, a substantially U-shape, or a rounded V-shape. In some embodiments, when the subassemblies 101, 103 are viewed in cross-section, the flexible electrical connector 108 may be adapted to be deformed to have a void between the flexible electrical connector 108 and the subject's body (e.g., FIGS. 3A, 3B).

The flexible electrical connector 108 may provide an adjustable distance S between the first and the second electrode subassemblies 101, 103. The flexible electrical connector 108 may be bent such that the first and the second electrode subassemblies 101, 103 are close together, but not touching (see, e.g., FIGS. 3A and 3B). When bent, the flexible electrical connector 108 may form a tent-like shape where the apex points away from the subject, and thus, the flexible electrical connector 108 may deform to a substantially concave shape away from the subject's body. The “tenting” of the flexible electrical connector 108 may allow for improved heat dissipation. The flexible electrical connector 108 may be pulled taught such that the first and the second electrode subassemblies 101, 103 are at a maximum distance, measured along the surface of the transducer apparatus 100A.

The flexible electrical connector 108 may have a length between the first and the second electrode subassemblies 101, 103 of at least 0.05 cm, or at least 0.1 cm, and not more than 5.00 cm. When viewed in a direction perpendicular to and toward the back face of the subassemblies 101, 103 (as in FIG. 1A), the flexible electrical connector 108 may provide a distance S between the first and the second electrode subassemblies 101, 103. The portion 138 of the flexible PCB 106 may connect the first and the second electrode subassemblies 101, 103 across the distance S. Due to the flexible electrical connector 108 being flexible, the distance S between the first and the second electrode subassemblies 101, 103 may vary as the subject moves (see, e.g., the discussion below regarding FIGS. 2C and 2D). The flexible electrical connector 108 may provide a maximum distance S between the first and the second electrode subassemblies 101, 103 when the flexible electrical connector 108 is planar. In some embodiments, one or both of the first and the second electrode subassemblies 101, 103 may comprise a non-adhesive conductive skin-contact layer. For example, the electrode subassemblies may comprise a non-adhesive conductive silicone skin-contact layer positioned on the front side of the anisotropic material layer 110, wherein the layer of conductive silicone has a non-adhesive front face that is textured in a manner that makes the front face of the layer of conductive silicone adhere to human skin. The non-adhesive skin-contact layer may be a mechanical adhesive layer having a suction-type quality to allow for a momentary release and re-adhesion of the subassemblies in the event that movement or twisting of the body creates stresses at the skin-subassembly interface. Such mechanical adhesives may utilize microstructures or nanostructures that function similarly to the setae and spatulae found on the foot pads of certain reptiles, which may allow for relatively strong adhesion to a wide range of surfaces. The microstructures may be arranged in patterns that optimize surface area and contact with surfaces, thereby enhancing adhesion through van der Waals forces. The adhesive structures may be fabricated from materials such as polymers (e.g., elastomers) or composites, which may provide flexibility and durability. The non-adhesive conductive silicone skin-contact material may be a silicone polymer (e.g., elastomer) with conductive particles (e.g., carbon particles) disposed therein. Such non-adhesive conductive silicone skin-contact material (i.e., mechanical adhesives) may be available as ElectroSkin Gecko product (available from Nanoleq, 8153 Rümlang, CH).

In some embodiments, the distance S may be subject-specific. For example, the size of the subject may be considered when determining the subject-specific distance S, and an appropriately-sized transducer apparatus may be selected from a variety of sizes (e.g., small, medium, and large).

In some embodiments, the flexible electrical connector 108 may have a thickness T (between the back face and the front face, depicted in FIG. 1C) and a width W (depicted in FIG. 1B) that is greater than the thickness. For example, the width W of the flexible electrical connector 108 may be at least 5 times greater than the thickness T of the flexible electrical connector 108. In some embodiments, the width W of the flexible electrical connector 108 may be at least 0.5 centimeter, or at least 1 centimeter, or at least 2 centimeters, or at least 5 centimeters. It is contemplated that the flexible electrical connector 108 having such a width W may inhibit undesired twisting of the flexible electrical connector 108 that could affect the spacing between the first and the second electrode subassemblies 101, 103.

In some embodiments, the flexible electrical connector 108 may be attached to the first and the second electrode subassemblies 101, 103 to permit angular pivoting (or angular rotating). For example, the flexible electrical connector 108 may have a sufficient width W to permit angular pivoting (or angular rotating) of the first and the second electrode subassemblies 101, 103 about an axis that extends out of the page in FIG. 1A.

The transducer apparatus 100A may include one or more blank spaces 126 (or void spaces), which do not overlap with any of the electrode elements 102 and include only the anisotropic material layer 110. Upon a rotational or translational shift of the subassembly, while (approximately) remaining in the preferred location with respect to treating a target in/on the subject's body, at least part of one or more of the blank spaces 126 may provide a relief region for the subject since no electrode elements 102 are in the blank spaces 126.

The transducer apparatus 100A may include the anisotropic material layer 110, which may be directly or indirectly electrically coupled to the plurality of electrodes 102 and located on a front face 128 (FIG. 1D) of the electrodes 102 configured to face the subject's body. The anisotropic material layer 110 may take any of the forms and include any of the features described in further detail herein with reference to the anisotropic material layer 110, 110B, 110C, and 110D of FIGS. 1D, 1E, 1F, and 1G, respectively.

The anisotropic material layer 110 may be disposed over (and in front of) the plurality of electrode elements 102 such that the anisotropic material layer 110 covers the electrode elements 102 and, optionally, the at least one blank space 126. In some embodiments, the anisotropic material layer 110 may not extend outward all the way to the edge of the substrate 104.

The transducer apparatus 100A may further include a foam material layer 124 directly or indirectly coupled to the anisotropic material layer 110, located on a front face 112 of the anisotropic material layer 110, and configured to contact the subject's body. In some embodiments, a layer of conductive adhesive material or conductive hydrogel may be located on the front face 112 of the anisotropic material layer 110 and may act as a biocompatible skin-contact layer. A front face 148 of the foam material layer 124 may include an adhesive to aid in securing the transducer apparatus 100A to the subject's skin. When viewed in a direction perpendicular to and toward the front face of the subassemblies 101, 103 (as in FIG. 1B), the foam material layer 124 may have an empty center such that the foam material layer 124 frames the anisotropic material layer 110. In some embodiments, the foam material layer 124 may be a continuous layer that covers some or most of the front face 112 of the anisotropic material layer 110 but does not cover all of the front face 112 of the anisotropic material layer 110. In some embodiments, the anisotropic material layer 110 may not extend outward all the way to the edge of the foam material layer 124. The foam material layer 124 may cover at most 5%, or at most 10%, or at most 20%, or at most 30%, or at most 40%, or at most 50%, or any percentages therebetween, of the anisotropic material layer 110.

When viewed in a direction perpendicular to and toward the front face 112 of the anisotropic material layer 110 of each subassembly 101, 103, the foam material layer 124 may include a perimeter 146 covering a perimeter of the anisotropic material layer 110 (FIGS. 1B, 1C, 1D). In some embodiments, the perimeter 146 of the foam material layer 124 may cover up to the edge of the perimeter of the anisotropic material layer 110. In some embodiments, the perimeter 146 of the foam material layer 124 may be larger than the perimeter of the anisotropic material layer 110. The perimeter 146 of the foam material layer 124 may have substantially the same shape as the perimeter of the anisotropic material layer 110. In some embodiments, an offset distance P between the perimeter 146 of the foam material layer 124 and the perimeter of the anisotropic material layer 110 (as illustrated in FIG. 1C) may be the same about the perimeter of the subassemblies 101, 103. Alternatively, the offset distance P between the perimeter of the anisotropic material layer 110 and the perimeter portion 146 of the foam material layer 124 (as in FIG. 1C) may vary about the perimeter of the subassemblies 101, 103.

The foam material layer 124 may facilitate a comfortable surface against the subject's body. In some embodiments, a transducer 100A having the anisotropic material layer 110 may include the foam material layer 124 to provide a conformable material in contact with the subject's body. The foam material layer 124 may be in the same plane as the conductive adhesive material 116 so that the electrode element 102 is placed as close as possible to the subject's skin, without the area of the front face 128 of the electrode element 102 of the transducer apparatus 100A projecting past the foam material layer 124. A back face 143 of the foam material layer 124 may not be in contact with the flexible electrical connector 108 (FIG. 1C).

In some embodiments, the transducer 100A may include a foam material layer 136 disposed on the front face 120 of the flexible electrical connector 108 and another foam material layer 134 disposed on the back face 122 of the flexible electrical connector 108 (as illustrated in FIG. 1C). In some embodiments, the foam material layer 124 may cover the front face 120 of the portion 138 of the flexible PCB 106. The foam material layer 136 on the front face 120 of the flexible electrical connector 108 may be a portion of the foam material layer 124 or may be separate from the foam material layer 124. In some embodiments, the foam material layer 136 and the foam material layer 124 may be separated from each other and may be neither a contiguous nor a unitary body. As such, when the flexible electrical connector 108 is deformed in a concave shape or “tented,” a back face 143 of the foam material layer 124 is not in contact with the flexible electrical connector 108. The transducer apparatus 100A may be more pliable against a subject's body when the flexible electrical connector 108 is free of contact with the back face 143 of the foam material layer 124.

The foam material layers 134, 136 may provide a comfortable surface against the subject's body. During certain treatments, the electrode subassemblies 101, 103 may need to be spaced apart at the maximum distance permitted by the length of the flexible electrical connector 108. As such, the flexible electrical connector 108 will be substantially flat and in contact or close to contact with the subject's body. The foam material layer 134, 136 may be included to provide a comfortable material in contact with the subject's body and/or to prevent rubbing of the flexible electrical connector 108 against the subject's body.

In some embodiments, the foam material layers 124, 134, and 136 may be at least 2 mm and at most 10 mm thick, or at least 5 mm and at most 10 mm thick, or at least 8 mm and at most 10 mm thick. In some embodiments, the foam material layer 124, 134, 136 may be formed at least partially of a soft material with airy open space. In some embodiments, the foam material layer 124, 134, 136 may be formed of a non-porous material to improve sterilizability and cleanability. In some embodiments, the foam material layer 124, 134, 136 may comprise one or more of low-density polyethylene (LDPE), silicone, polyurethane, or ethylene-vinyl acetate (EVA), each of which may be an open-cell, closed-cell, or partially open/closed-cell foam. In some embodiments, a closed-cell foam is preferred. In some embodiments, the foam may be an adhesive coated foam. In some embodiments, the foam may be a 3M™ Tegaderm™ product (3M, Saint Paul, MN, USA), which may include adhesive on one side facing the patient's body and facing away from the substrate (104).

The substrate 104 may be configured for attaching the transducer 100A to a subject's body and may be configured to secure the various components of the transducer 100A. The substrate 104 may hold the at least one electrode element 102 and the anisotropic material layer 110 against the subject's body. The substrate 104 of each subassembly 101, 103 may include a front face 104(F) facing the subject's body and a back face 104(B) opposite the front face 104(F) (FIGS. 1C, 1D). The front face 104(F) of the substrate 104 may face the at least one electrode element 102 and the anisotropic material layer 110. The substrate 104 of each subassembly 101, 103 may have an outer perimeter extending beyond an outer edge of the respective anisotropic material layer 110 of each subassembly. The substrate 104 of each subassembly 101, 103 may be contoured to match a shape of the outer edge of each respective anisotropic material layer 110. The substrate 104 of each subassembly 101, 103 may have an outer perimeter extending beyond the outer edge of all other components of each subassembly, such that an adhesive on the front face 104(F) of the substrate 104 adheres the substrate 104 to the subject's body, thereby holding each subassembly against the subject's body. The substrates 104 of the first and second electrode subassemblies 101, 103 may be separated by the flexible electrical connector 108. Accordingly, in some embodiments, the substrates 104 of the first and second electrode subassemblies 101, 103 are distinct and not a unitary body.

Each of the subassemblies 101, 103 may have separated portions of the substrate 104. In some embodiments, the flexible electrical connector 108 may not include a portion of the substrate 104. In some embodiments, the substrate 104 may not directly contact the subject's skin due to the foam material layer 148. Suitable materials for the substrate 104 may include, for example, tape, bandage, cloth, nonwoven fabric, foam, flexible plastic, and/or a conductive medical gel. The transducer 100A may be affixed to the subject's body via the substrate 104 (e.g., via an adhesive layer and/or a conductive medical gel). The substrate 104 may be an adhesive bandage. The substrate may aid in securing a cable (not shown) connected to the flexible PCB 106 for providing signals to the flexible PCB 106 for generating TTFields with the transducer apparatus 100A.

As discussed above, the transducer apparatus 100A may further include the anisotropic material layer 110. As shown, the anisotropic material layer 110 has a front face 112 and a back face 114, wherein the back face 114 faces the array of electrode elements 102 (as illustrated in FIG. 1D). The anisotropic material layer 110 has anisotropic thermal properties and/or anisotropic electrical properties. If the anisotropic material layer 110 has anisotropic thermal properties (for example, greater thermal conductivity in the plane of the layer than through the plane of the layer), then the layer spreads the heat out more evenly over a larger surface area. If the anisotropic material layer 110 has anisotropic electrical properties (for example, greater electrical conductivity in the plane of the layer than through the plane of the layer), then the layer spreads the current out more evenly over a larger surface area. In each case, this lowers the temperature of the hot spots and raises the temperature of the cooler regions when a given AC voltage is applied to the array of electrode elements. Accordingly, the current may be increased (thereby increasing the therapeutic effect) without exceeding the safety temperature threshold at any point on the subject's skin.

In some embodiments, the anisotropic material layer 110 may be anisotropic with respect to electrical conductivity properties. In some embodiments, the anisotropic material layer 110 may be anisotropic with respect to thermal conductivity properties. In some embodiments, the anisotropic material layer 110 may be anisotropic with respect to both electrical conductivity properties and thermal conductivity properties.

The anisotropic thermal properties may include directional thermal properties. Specifically, the anisotropic material layer 110 may have a first thermal conductivity in a direction that is perpendicular to its front face (skin-facing surface) 112 that is different from a second thermal conductivity of the anisotropic material layer 110 in directions that are parallel to the front face 112. For example, the second thermal conductivity of the anisotropic material layer 110 may be more than two times higher than the first thermal conductivity. In some preferred embodiments, the second thermal conductivity may be more than ten times higher than the first thermal conductivity. For example, the second thermal conductivity may be more than: 1.5 times, 2 times, 3 times, 5 times, 10 times, 20 times, 100 times, 200 times, or even more than 1,000 times higher than the first thermal conductivity.

The anisotropic electrical properties include directional electrical properties. Specifically, the anisotropic material layer 110 may have a first electrical conductivity (or, conversely, resistance) in a direction that is perpendicular to its front face 112 that is different from a second electrical conductivity (or resistance) of the anisotropic material layer 110 in directions that are parallel to the front face 112. For example, the second resistance may be less than the first resistance. In some embodiments, the second resistance may be less than half of the first resistance or less than 10% of the first resistance. For example, the second resistance of the anisotropic material layer 110 may be less than: 75%, 50%, 40%, 30%, 20%, 10%, 5%, 1%, 0.5%, or even less than 0.1% of the first resistance.

In some embodiments (e.g., when the anisotropic material layer 110 is a sheet of pyrolytic graphite), the anisotropic material layer 110 may have both anisotropic electrical properties and anisotropic thermal properties.

The anisotropic material layer 110 may comprise graphite (e.g., a sheet of graphite or a graphite sheet). Examples of suitable forms of graphite include synthetic graphite, such as pyrolytic graphite (including, but not limited to, Pyrolytic Graphite Sheet (PGS), available from Panasonic Industry, Kadoma, Osaka, Japan), other forms of synthetic graphite, including but not limited to, graphite foil made from compressed high purity exfoliated mineral graphite (including, but not limited to, that supplied as MinGraph® 2010A Flexible Graphite, available from Mineral Seal Corp., Tucson, Arizona, USA), or graphitized polymer film, e.g., graphitized polyimide film, (including, but not limited to, that supplied by Kaneka Corp., Moka, Tochigi, Japan). In alternative embodiments, conductive anisotropic materials other than graphite may be used instead of graphite.

In some embodiments, the anisotropic material layer 110 is a sheet of pyrolytic graphite. Thermal conductivity of pyrolytic graphite sheets in directions that are parallel to the front face 112 of those sheets is typically more than 50 times higher than the thermal conductivity of those sheets in directions that are perpendicular to the front face 112. Electrical resistivity of pyrolytic graphite sheets in directions that are parallel to the front face 112 of those sheets may typically be less than 2% of the electrical resistivity of those sheets in directions that are perpendicular to the front face 112.

The transducer 100A may further include at least one layer of conductive adhesive material 116 and/or hydrogel disposed on a front facing side 112 of the anisotropic material layer 110. In some embodiments, the at least one layer of conductive adhesive material 116 may be disposed on the front face 112 of the anisotropic material layer 110. The at least one layer of conductive adhesive material 116 may have a biocompatible front surface. In some embodiments, there may be only a single layer of conductive adhesive material 116, and that single layer (the front layer) may be biocompatible. In some embodiments, there may be more than one layer of conductive adhesive material 116, in which case only the front layer may be biocompatible, or the front layer and one or more other layers may be biocompatible. In some embodiments, the front layer of conductive adhesive material 116 may be configured to ensure good electrical contact between the device and the body. In some embodiments, the front layer of conductive adhesive material 116 may be configured to ensure good adhesion between the anisotropic material layer 110 and the foam material layer 124. In some embodiments, the front layer of conductive adhesive material 116 may cover the entire front face 112 of the anisotropic material layer 110. The front layer of conductive adhesive material 116 may be the same size or larger than the anisotropic material layer 110. In some embodiments, a front face of conductive adhesive material 116 may comprise hydrogel. In these embodiments, the hydrogel may have a thickness between 50 and 2,000 μm. In some embodiments, the front layer of conductive adhesive material 116 may comprise a conductive adhesive composite as further disclosed herein.

The transducer 100A may further include at least one layer of conductive material 118, such as a conductive adhesive material or hydrogel, disposed on a back facing side 114 of the anisotropic material layer 110. The conductive material 118 may be positioned between the array of electrode elements 102 and the back face 114 of the anisotropic material layer 110 facing the array (as illustrated in FIG. 1D). The conductive material 118 may facilitate the electrical contact between the array of electrode elements 102 and the back face 114 of the anisotropic material layer 110. In some embodiments, the conductive material 118 may be a layer of conductive adhesive material. In some embodiments, the conductive material 118 may be a layer of hydrogel. In other embodiments, a different conductive material (e.g., conductive grease, conductive adhesives, conductive tape, etc.) may be used. For example, the conductive material 118 may comprise a conductive adhesive composite as further disclosed herein.

In some embodiments, the flexible PCB 106 may be positioned between a back face of the conductive material 118 and the substrate 104. In some embodiments, the electrode elements 102 may be positioned between the back face of the conductive material 118 and the substrate 104. In some embodiments, the front faces of the electrode elements 102 may be in direct contact with the back face of the conductive adhesive 118. In some embodiments, the foam material layer 124 may be positioned between a front face 130 of the conductive adhesive material 116 and the subject's body.

In some embodiments, the at least one layer of conductive adhesive material 116 and/or the layer of conductive material 118 may be a single layer of non-hydrogel conductive adhesive such as the developmental product FLX068983—FLEXcon® OMNI-WAVE™ TT 200 BLACK H-502 150 POLY H-9 44PP-8 from FLEXcon, Spencer, MA, USA, or other such OMNI-WAVE products from FLEXcon; or ARcare® 8006 electrically conductive adhesive composition manufactured and sold by Adhesives Research, Inc. (Glen Rock, PA, USA). Non-hydrogel conductive adhesives may comprise a waterless polymer with adhesive properties and carbon particles, powder, fibers, flakes, granules and/or nanotubes. The adhesive polymer may be, for example, an acrylic polymer or a silicone polymer, or combination thereof, which may be available as acrylic-or silicone-based carbon-filled adhesive tapes. The adhesive may additionally include one or more conductive polymers (such as, for example, polyaniline (PANI), or poly(3,4-ethylenedioxythiophene) (PEDOT), or others known in the art). The conductive filler in the at least one layer of conductive adhesive material 116 or conductive material 118 may be non-metallic. In these embodiments, the conductive adhesive may have a thickness between 10 and 2,000 μm, such as, from 20 to 1,000 μm, or 30 to 400 μm.

In some embodiments, the transducer 100A may be constructed using a pre-formed 3-(or more) layer laminate comprising the conductive material 118, the anisotropic material layer 110, and the at least one layer of conductive adhesive material 116. In some embodiments, the at least one conductive adhesive material 116 and the conductive material 118 may both be conductive adhesive composites as described above, and the anisotropic material layer 110 may be a thin sheet of synthetic graphite such as pyrolytic graphite, as described above. In some embodiments, the at least one conductive adhesive material 116 and the conductive material 118 may be the same material or may be different. By way of example, in some embodiments, both the conductive adhesive material 116 and the conductive material 118 may comprise an acrylic polymer and a carbon powder filler; or both the conductive adhesive material 116 and the conductive material 118 may comprise an acrylic polymer and a carbon fiber filler. In some embodiments, the conductive adhesive material 116 comprises an acrylic polymer and a carbon fiber filler, and the conductive material 118 comprises an acrylic polymer and a carbon powder filler; or vice-versa. In some embodiments, one or both of the conductive adhesive material 116 and the conductive material 118 may be a hydrogel.

FIG. 1E depicts a cross-section view of an example transducer apparatus 100B according to some embodiments. The transducer apparatus 100B in FIG. 1E is the same as the transducer apparatus 100A in FIG. 1C, except for the flexible electrical connector 108B. The parts in FIG. 1E use the same reference numbering as the parts in FIG. 1C, except for the added suffix of “B”. In FIG. 1E, the flexible electrical connector 108B includes foam material layer 136B disposed on the front surface 120B and does not include a foam material layer disposed on the back surface 122B. The foam material layer 136B may facilitate a comfortable surface against the subject's skin. The flexible electrical connector 108B may have greater flexibility and an ability to flex in a concave direction by including only one foam material layer.

FIG. 1F depicts a cross-section view of an example transducer apparatus 100C according to some embodiments. The transducer apparatus 100C in FIG. 1F is the same as the transducer apparatus 100A in FIG. 1C, except for the flexible electrical connector 108C. The parts in FIG. 1F use the same reference numbering as the parts in FIG. 1C, except for the added suffix of “C”. In FIG. 1F, the flexible electrical connector 108C does not include foam material layers on either the front face 120C or the back face 122C. Without the foam material layers on the front and the back faces 120C, 122C of the flexible electrical connector 108C, the flexible electrical connector 108C may have a greater range of flexibility.

FIG. 1G depicts a cross-section view of an example transducer apparatus 100D according to some embodiments. The transducer apparatus 100D in FIG. 1G is the same as the transducer apparatus 100A in FIG. 1C, except for the flexible electrical connector 108D. The parts in FIG. 1G use the same reference numbering as the parts in FIG. 1C, except for the added suffix of “D”. In FIG. 1G, the anisotropic material layer 110D may be connected (via conductive material layer 118D) to the front face 120D of the flexible electrical connector 108D. As such, the anisotropic material layer 110D on the front face 120D of the flexible electrical connector 108D forms a unitary body with the anisotropic material layers 110D of the subassemblies 101D, 103D.

In FIG. 1G, the flexible electrical connector 108D includes portion 138D of the flexible PCB 106D and additionally includes a portion of the conductive adhesive material 118D, a portion of the anisotropic material layer 110D, a portion of the conductive adhesive material layer 116D, and a portion of the foam material layer 124D. As such, these portions bridge the distance S between the two subassemblies 101, 103. Extending across (or spanning) the distance S, the conductive material 118D contacts the front face 120D of the portion 138D of the flexible PCB 106D and contacts the back face 114D of the anisotropic material layer 110D. Extending across (or spanning) the distance S, the back face 114D of the anisotropic material layer 110D contacts the conductive material 118D, and the front face 112D of the anisotropic material layer 110D contacts the conductive adhesive material layer 116D. Extending across (or spanning) the distance S, the conductive adhesive material layer 116D contacts the front face 112D of the anisotropic material layer 110D, and the front face 130D of the conductive adhesive material layer 116D contacts the foam material layer 124D.

FIG. 2A depicts a top plan view of an example transducer apparatus according to some embodiments. FIG. 2A illustrates the transducer apparatus 200A as viewed in a direction perpendicular to and toward a back face of the transducer apparatus 200A (i.e., the side facing away from the subject's body). The transducer apparatus 200A in FIG. 2A is substantially the same as the transducer apparatus 100A in FIGS. 1A to 1D except for the shape of the subassemblies, the shape of the electrode elements, and the shape of the flexible PCB, and in other respects has a similar labelling convention to that in FIGS. 1A to 1D (FIGS. 2A to 2D use a 2XXA to 2XXD labelling notation in place of the 1XX labelling notation of FIGS. 1A to 1D). The transducer apparatus 200A includes first and second electrode subassemblies 201A, 203A with an egg shape, oval shape, ovaloid, or ovoid shape, or asymmetric oval, ovaloid, or ovoid shape. The first and the second electrode subassemblies 201A, 203A are oriented relative to each other such that a middle 246A(M) of smaller end 246A of the first and the second electrode subassemblies 201A, 203A are closer to each other than a middle 248A(M) of larger end 248A, where the larger end 248A is opposite to the smaller end 246A. The flexible electrical connector 208A may couple and extend across the first and the second electrode subassemblies 201A, 203A, and, in some embodiments, it may couple and extend across the first and the second electrode subassemblies 201A, 203A closer to the larger end 248A (as shown in FIG. 2A).

In FIG. 2A, the transducer apparatus 200A may include a plurality of electrode elements 202A (i.e., 202A(1), 202A(2), 202A(3), 202A(4)) (shown in dashed lines). The electrodes 202A may be disposed on an outward facing side (i.e., the back face or top) of the anisotropic material layer 210A and on the skin-facing side (i.e., the front face or bottom) of the flexible PCB 206A. As shown in FIG. 2A, the electrode elements 202A may have a triangular shape, substantially triangular shape, a rounded-triangular shape, or substantially rounded-triangular shape. The first and the second electrode subassemblies 201A, 203A may include two electrode elements, respectively. The two electrode elements may be disposed in line with each other, with one electrode element positioned near the top end 246A and the other electrode element positioned near the bottom end 248A.

FIG. 2B depicts a top plan view of an example transducer apparatus according to some embodiments. FIG. 2B illustrates the transducer apparatus 200B as viewed in a direction perpendicular to and toward a back face of the transducer apparatus 200B (i.e., the side facing away from the subject's body). The transducer apparatus 200B in FIG. 2B is substantially the same as the transducer apparatus 200A in FIG. 2A except for the number and the shape of the electrode elements, and in other respects has a similar labelling convention to that in FIG. 2A. In FIG. 2B, the transducer apparatus 200B has a plurality of electrode elements 202B (i.e., 202B(1), 202B(2)) (shown in dashed lines). The electrode elements 202B may be disposed on an outward facing side (i.e., the back face or top) of an anisotropic material layer 210B and on the skin-facing side (i.e., the front face or bottom) of the flexible PCB 206B. As shown in FIG. 2B, the electrode elements 202B may have a circular shape, substantially circular shape, oval shape, or substantially oval shape. The first and the second electrode subassemblies 201B, 203B may each include one electrode element 202B, respectively, positioned near a top end 246B or a bottom end 248B.

FIG. 2C depicts a top plan view of an example transducer apparatus according to some embodiments. FIG. 2C illustrates the transducer apparatus 200C as viewed in a direction perpendicular to and toward a back face of the transducer apparatus 200C (i.e., the side facing away from the subject's body). The transducer apparatus 200C in FIG. 2C is substantially the same as the transducer apparatus 200A in FIG. 2A except for the shape of the subassemblies, the shape of the electrodes, the shape of the flexible PCB, and the flexible electrical connector, and in other respects has a similar labelling convention to that in FIG. 2A. Further, similar to the embodiments depicted in FIGS. 1E, 1F, and 1G, the transducer apparatus 200C in FIG. 2C does not include a foam material on the back face 222C of the electrical connector 208C. As such, the portion 238C of the electrical connector 208C is visible in FIG. 2C.

In FIG. 2C, the transducer apparatus 200C includes first and second electrode subassemblies 201C, 203C with a triangular shape, substantially triangular shape, rounded-triangular shape, substantially rounded-triangular shape, or trilobal shape. The first and the second electrode subassemblies 201C, 203C are oriented relative to each other such that respective middle points 248C(M) of larger end 248C of the first and the second electrode subassemblies 201C, 203C are further apart from each other than respective middle points 246C(M) of smaller end 246C of the first and the second electrode subassemblies 201C, 203C, where the smaller end 246C may be opposite the larger end 248C. The flexible electrical connector 208C may couple and extend across the first and the second electrode subassemblies 201C, 203C, for example, substantially in the middle of the first and the second electrode subassemblies (as shown in FIG. 2C). The flexible electrical connector 208C may have a U-shape, substantially U-shape, or rounded V-shape in the same plane as the first and the second electrode subassemblies 201C, 203C. An apex 208C(A) of the flexible electrical connector 208C may point, for example, to the larger ends 248C (as shown in FIG. 2C), or may, alternatively, may point to the smaller ends 246C.

In FIG. 2C, the transducer apparatus 200C may include a plurality of electrode elements 202C (i.e., 202C(1), 202C(2), 202C(3), 202C(4)) (shown in dashed lines). The electrode elements 202C may be disposed on an outward facing side (i.e., the back face or top) of an anisotropic material layer 210C and on the skin-facing side (i.e., the front face or bottom) of the flexible PCB 206C. As shown in FIG. 2C, the electrode elements 202C may have a triangular shape, substantially triangular shape, rounded-triangular shape, substantially rounded-triangular shape, oval shape, substantially oval shape, ovoid shape, or substantially ovoid shape, or asymmetric oval, ovaloid, or ovoid shape. Each electrode element may define a tapered end. The first and the second electrode subassemblies 201C, 203C may each include two electrode elements, respectively. The two electrode elements 202C(1), 202C(2) and 202C(3), 202C(4) may be disposed adjacent to each other and positioned substantially in the center of the first and the second electrode subassemblies 201C, 203C, respectively.

FIG. 2D depicts a top plan view of an example transducer apparatus according to some embodiments. FIG. 2D illustrates the transducer apparatus 200D as viewed in a direction perpendicular to and toward a back face of the transducer apparatus 200D (i.e., the side facing away from the subject's body). The transducer apparatus 200D in FIG. 2D is substantially the same as the transducer apparatus 200C in FIG. 2C except for the flexible electrical connector 208D, and in other respects has a similar labelling convention to that in FIG. 2C. Further, similar to the embodiments depicted in FIGS. 1E, 1F, 1G, and 2C, the transducer apparatus 200D in FIG. 2D does not include a foam material on the back face 222D of the electrical connector 208D. As such the portion 238D of the electrical connector 208D is visible in FIG. 2D.

The flexible electrical connector 208D may couple and extend across the first and the second electrode subassemblies 201D, 203D toward the larger end 248D of the first and the second electrode subassemblies (as shown in FIG. 2D), although other connection locations are also possible. The flexible electrical connector 208D may have a U-shape, substantially U-shape, or rounded V-shape in the same plane as the first and the second electrode subassemblies 201D, 203D. An apex 208D(A) of the flexible electrical connector 208D may point to the smaller ends 246D (as shown in FIG. 2D), or may, alternatively, point to the larger ends 248D.

In some embodiments, when viewed in a direction perpendicular to and toward the back face of the subassemblies 101, 103, the flexible electrical connector 108 may have a different shape, such as, for example: a U-shape, substantially U-shape, or rounded V-shape (e.g., FIGS. 2C, 2D); a shape with two or more bends, such as an undulating shape, serpentine shape, wavelike shape, zig-zag shape, or the like; a shape with a combination of concave and convex portions; or a spiral shape. In some embodiments, due to these various possible shapes, the subassemblies 101, 103 may be translatable, rotatable, and/or rotatable relative to each other.

FIG. 3A to 3D depict a perspective view of an example transducer apparatus on a mannequin, according to some embodiments.

In FIG. 3A, the transducer apparatus 100A of FIGS. 1A to 1D is depicted placed over the sternum area of a mannequin, and the target location (i.e. tumor location) for the TTFields therapy may be in the torso. For illustration purposes, the two electrode subassemblies 101, 103 are shown with the flexible PCB 106, the anisotropic material layer 110, and the foam layer 124 and without the substrate 104 (which would obscure these features). The two electrode subassemblies 101, 103 are spaced apart by the flexible electrical connector 108. As placed on the mannequin, the flexible electrical connector 108 includes a bend away from the subject and forms a substantially concave shape away from the subject. In some embodiments, a flexible material (such as foam wedges) may be attached or inserted on the underside of the flexible electrical connector 108 (i.e., between the flexible electrical connector 108 and the subject's body) in order to hold the flexible electrical connector 108 off the subject's body. In some embodiments, when applying TTFields treatment, at least one pair of transducer apparatuses are employed. For example, as shown in FIG. 3A, the first transducer apparatus 100A may be positioned on the subject's chest, and a second transducer apparatus of the first pair of transducer apparatuses may be positioned on the subject's back (not shown). Optionally, a second pair of transducer apparatuses may be positioned on the left and right sides of the subject's torso. For example, transducer apparatus 300A, partly obscured, is shown located on one side of the subject's torso, which could be paired with a fourth transducer apparatus on the other side of the subject's torso (not shown).

In FIG. 3B, the transducer apparatus 200A of FIG. 2A is depicted placed over the a sternum area of a mannequin, and the target location (i.e. tumor location) for the TTFields therapy may be in the torso. For illustration purposes, the two electrode subassemblies 201A, 203A are shown with the flexible PCB 206A, the anisotropic material layer 210A, and the foam layer 224A and without the substrate 204A (which would obscure these features). The two electrode subassemblies 201A, 203A are spaced apart by the flexible electrical connector 208A. As placed on the mannequin, the flexible electrical connector 208A includes a bend away from the subject and forms a substantially concave shape away from the subject. In some embodiments, when applying TTFields treatment, at least one pair of transducer apparatuses are employed. As discussed above with respect to FIG. 3A, and similarly for FIG. 3B, the first transducer apparatus 200A may be paired with a second transducer apparatus on the subject's back (not shown). Optionally, another pair of transducer apparatuses may be positioned on the left and right side of the subject's torso (one of which, 300B, is partially visible in FIG. 3B).

In FIG. 3C, example transducer apparatuses 300C and 300C′ are depicted placed on a mannequin, and the target location (i.e. tumor location) for the TTFields therapy may be in the torso (transducer apparatus 300C′ is shown in dashed lines to indicate that it is located on the backside of the mannequin). The transducer apparatuses 300C and 300C′ are substantially the same as transducer apparatus 100A in FIGS. 1A to 1D, except for the shape of the transducer apparatuses and the shape of the flexible electrical connectors. The transducer apparatus 300C includes first and second electrode subassemblies 301C, 303C each with a c-shape, substantially c-shape, jelly-bean or kidney bean shape, substantially jelly-bean or kidney bean shape, bent elbow shape, or substantially bent elbow shape. The transducer apparatus 300C′ includes first and second electrode subassemblies 301C′, 303C′ each with a triangular, substantially triangular, rounded triangular, substantially rounded triangular, or trilobal shape. For the transducer apparatus 300C, the first electrode subassembly 301C may be positioned substantially just below a subject's arm-pit area to target a tumor or a cluster of cancer cells in the sentinel lymph node, for example. The second electrode subassembly 303C may then be positioned on or about the subject's mammary/pectoral muscle. Additionally, for the transducer apparatus 300C′, the electrode subassembly 301C′ may be positioned on the front of the subject's torso, for example on the upper chest. The second electrode subassembly 303C′ (shown in dashed line) may then be positioned on a back of the subject's torso, for example the shoulder-blade area. The flexible electrical connectors 308C, 308C′ may be of any shape/length/width; for example, they may be wider and/or longer than the flexible electrical connectors described above. Although the two subassemblies for each of the transducer apparatuses 300C and 300C′ are depicted as having the same size and shape, they need not be. Further, the shapes and sizes of the subassemblies used in the first transducer apparatus may be the same or may be different to those of the other transducer apparatus. Transducer apparatuses 300C and 300C′ may operate as a pair; for example, an electric field may be induced between transducer apparatuses 300C and 300C′. In some embodiments, one transducer apparatus (either 300C or 300C′) of a pair of transducer apparatuses comprises two electrode subassemblies and the other of the pair of transducer apparatuses may be a single transducer array (having one electrode subassembly).

In FIG. 3D, example transducer apparatuses 300D and 300D′ are depicted placed on a mannequin, and the target location (i.e. tumor location) for the TTFields therapy may be in the torso (one electrode subassembly of the transducer apparatus 300D′ is shown in dashed lines to indicate that it is located on the backside of the mannequin). The transducer apparatuses 300D and 300D′ are substantially the same as transducer apparatus 100A in FIGS. 1A to 1D, except for the shape of the transducer apparatuses and the shape of the flexible electrical connectors. Transducer apparatuses 300D and 300D′ may operate as a pair in the manner described for transducer apparatuses 300C and 300C′, above. The transducer apparatus 300D includes first and second electrode subassemblies 301D, 303D each with a c-shape, substantially c-shape, jelly-bean or kidney bean shape, substantially jelly-bean or kidney bean shape, bent elbow shape, or substantially bent elbow shape. The transducer apparatus 300D′ includes first and second electrode subassemblies 301D′, 303D′ with an oval, ovaloid, ovoid, or circular shape or a substantially oval, substantially ovaloid, substantially ovoid, or substantially circular shape. For the transducer apparatus 300D, the first electrode subassembly 301D may be positioned substantially just below a subject's arm-pit area to target a tumor or a cluster of cancer cells in the sentinel lymph node, for example. The second electrode subassembly 303D may (or may not) be similarly shaped and may be positioned on or about the subject's mammary/pectoral muscle. Additionally, the first electrode subassembly 301D′ may be positioned on the front of the subject's torso, for example on the upper chest. The second electrode subassembly 303D′ (shown in dashed line) may then be positioned on a back of the subject's torso, for example the shoulder-blade area. Although the two subassemblies for each of the transducer apparatuses 300D and 300D′ are depicted as having the same size and shape, they need not be. Further, the shapes and sizes of the subassemblies used in the first transducer apparatus may be the same or may be different to those of the other transducer apparatus. The flexible electrical connectors 308D, 308D′ may be of any shape/length/width; for example, they may be or may include wires to electrically connect the electrode subassemblies. In some embodiments, one transducer apparatus (either 300D or 300D′) of a pair of transducer apparatuses comprises two electrode subassemblies and the other of the pair of transducer apparatuses may be a single transducer array (having one electrode subassembly).

It should be appreciated that the transducer apparatus 200B, 200C, and 200D may also be used in substantially the same manner as the transducer apparatus 100A, 200A, 300C, 300C′, 300D, and 300D′ as described above and in various combinations.

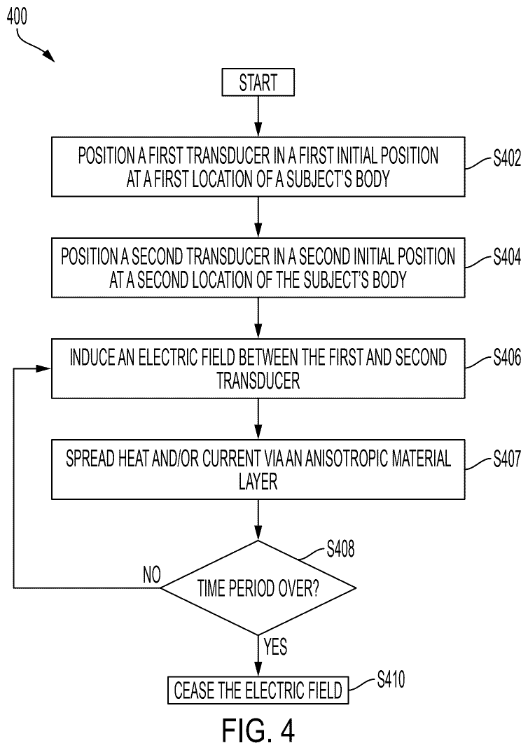

FIG. 4 is a flowchart depicting an example method 400 of applying TTFields to a subject's body according to some embodiments.

In step S402, the method 400 may include positioning a first transducer in a first initial position at a first location of the subject's body. The first transducer may be one of the example transducer apparatuses discussed herein (e.g., transducer apparatuses 100A, 200A, 200B, 200C, 200D, 200E, 300A, 300B, 300C, 300C′, 300D, 300D′). The first transducer may be affixed to the subject's body via an adhesive layer. The first transducer apparatus may include an anisotropic material layer electrically coupled to the plurality of electrodes and located between the plurality of electrodes and the subject's body, with the flexible electrical connector electrically linking the first electrode subassembly and the second electrode subassembly.

In some embodiments, both of the first and second electrode subassemblies of the first transducer may be positioned over one target region in or on the subject's body. For example, FIGS. 3A and 3B depict the first and second electrode subassemblies positioned over one target region in or on the subject's body, namely the sternum area. For example, the first and second electrode subassemblies are positioned on either side of the sternum.

In some embodiments, the first electrode subassembly of the first transducer may be positioned over a first target region in or on the subject's body, and the second electrode subassembly may be positioned over a second target region in or on the subject's body. In some embodiments, the first target region in or on the subject's body may be a first tumor, and the second target region in or on the subject's body may be a second tumor. In some embodiments, the first target region in or on the subject's body may be a tumor, and the second target region in or on the subject's body may be a sentinel lymph node. In some embodiments, the first target region in or on the subject's body may be a tumor in or on the subject's breast, and the second target region in or on the subject's body may be a sentinel lymph node in or around the subject's armpit. For example, FIGS. 3C and 3D depict the first electrode subassembly positioned over a breast to target a tumor in this region and depict the second electrode subassembly positioned near a subject's arm-pit area to target a tumor in the sentinel lymph node.

In step S404, the method 400 may include positioning a second transducer in a second initial position at a second location of the subject's body. The second transducer may be one of the example transducer apparatuses discussed herein (e.g., transducer apparatuses 100A, 200A, 200B, 200C, 200D, 200E, 300A, 300B, 300C, 300C′, 300D, 300D′). The second transducer may be affixed to the subject's body via an adhesive layer. The second transducer may include an anisotropic material layer electrically coupled to the plurality of electrodes and located between the plurality of electrodes and the subject's body, with the flexible electrical connector electrically linking the first electrode subassembly to the second electrode subassembly.

In step S406, the method 400 may include inducing an electric field between the first transducer located at the first location of the subject's body and the second transducer located at the second location of the subject's body.

In step S407, during inducing the electric field, the method 400 may include spreading heat and/or current via an anisotropic material layer from the plurality of electrodes in a plane perpendicular to a direction from the plurality of electrodes to the subject's body.

In step S408, the method 400 may include determining whether a first period of time has passed. Upon determining that the first period of time has passed, the method 400 proceeds to step S410. Otherwise, the method 400 returns to step S406. After inducing the electric field for more than the first period of time, the method 400 proceeds to step S410, which may include ceasing the electric field.

The invention includes other illustrative embodiments (“Embodiments”) as follows.

Embodiment 1. A transducer apparatus for delivering tumor treating fields to a subject's body, the transducer apparatus comprising: first and second electrode subassemblies, each subassembly configured to be positioned over the subject's body with a front face of the subassembly facing the subject's body, each subassembly having a back face opposite the front face, each subassembly comprising: at least one electrode element; an anisotropic material layer electrically coupled to the at least one electrode element, the anisotropic material layer comprising a front face facing the subject's body and a back face opposite the front face, the back face facing the at least one first electrode element; and a flexible electrical connector electrically connecting the first and second electrode subassemblies, wherein the first and second electrode subassemblies are separated by the flexible electrical connector.

Embodiment 2. The transducer apparatus of Embodiment 1, wherein each subassembly further comprises a substrate for holding the at least one electrode element and the anisotropic material layer against the subject's body, wherein the substrate of each subassembly comprises a front face facing the subject's body and a back face opposite the front face, the front face of the substrate facing the at least one first electrode element and the anisotropic material layer, and wherein the substrates of the first and second electrode subassemblies are separated by the flexible electrical connector.

Embodiment 3. The transducer apparatus of Embodiment 2, wherein the substrates of the first and second electrode subassemblies are distinct and not a unitary body.

Embodiment 4. The transducer apparatus of Embodiment 1, wherein the substrate of each subassembly has an outer perimeter extending beyond an outer edge of the respective anisotropic material layer of each subassembly.

Embodiment 5. The transducer apparatus of Embodiment 4, wherein the substrate of each subassembly is contoured to match a shape of the outer edge of each respective anisotropic material layer.

Embodiment 6. The transducer apparatus of Embodiment 1, wherein the flexible electrical connector electrically connects the at least one electrode element of the first electrode subassembly and the at least one electrode element of the second electrode subassembly.

Embodiment 7. The transducer apparatus of Embodiment 1, wherein when viewed in a direction perpendicular to and toward the back face of the subassemblies, the flexible electrical connector has a linear shape.

Embodiment 8. The transducer apparatus of Embodiment 1, wherein when viewed in a direction perpendicular to and toward the back face of the subassemblies, the flexible electrical connector has a U-shape, a substantially U-shape, or a rounded V-shape.

Embodiment 9. The transducer apparatus of Embodiment 1, wherein the flexible electrical connector is adapted to be deformed to a substantially concave shape away from the subject's body.

Embodiment 10. The transducer apparatus of Embodiment 1, wherein when the subassemblies are viewed in cross-section, the flexible electrical connector is adapted to be deformed from a substantially planar shape to a U-shape, a substantially U-shape, or a rounded V-shape.

Embodiment 11. The transducer apparatus of Embodiment 1, wherein when the subassemblies are viewed in cross-section, the flexible electrical connector is adapted to be deformed to have a void between the flexible electrical connector and the subject's body.

Embodiment 11A. The transducer apparatus of Embodiment 1, wherein one or more wedges reside between the flexible electrical connector and the subject's body.

Embodiment 11B. The transducer apparatus of Embodiment 11A, wherein the one or more wedge are comprised of foam material.

Embodiment 12. The transducer apparatus of Embodiment 1, wherein the flexible electrical connector comprises a front face facing the subject's body and a back face opposite the front face, wherein the front face of the flexible electrical connector is free of adhesive material.

Embodiment 12A. The transducer apparatus of Embodiment 1, wherein when the subassemblies are viewed in cross-section, the flexible electrical connector is adapted not to be adhesively held to the subject's body.

Embodiment 13. The transducer apparatus of Embodiment 1, wherein the flexible electrical connector comprises a flexible printed circuit board or portion thereof.

Embodiment 14. The transducer apparatus of Embodiment 1, wherein the flexible electrical connector comprises a front face facing the subject's body and a back face, wherein the transducer apparatus further comprises a foam layer on at least one of the front face or the back face of the flexible electrical connector.

Embodiment 15. The transducer apparatus of Embodiment 1, wherein the flexible electrical connector comprises a front face facing the subject's body and a back face, wherein the transducer apparatus further comprises an anisotropic material layer on the front face of the flexible electrical connector.

Embodiment 15A. The transducer apparatus of Embodiment 1, wherein the flexible electrical connector comprises a flexible PCB, or portion thereof, having a front face facing the subject's body and a back face, wherein the transducer apparatus further comprises an anisotropic material layer on the front face of the flexible PCB.

Embodiment 16. The transducer apparatus of Embodiment 15, wherein the anisotropic material layer on the front face of the flexible electrical connector forms a unitary body with respective anisotropic material layers of the subassemblies.

Embodiment 16A. The transducer apparatus of Embodiment 15A, wherein the anisotropic material layer on the front face of the flexible PCB forms a unitary body with respective anisotropic material layers of the subassemblies.

Embodiment 16B. The transducer apparatus of Embodiment 1, wherein a length of the flexible electrical connector between the subassemblies is at least 0.1 cm and no more than 5.00 cm.

Embodiment 17. The transducer apparatus of Embodiment 1, wherein when viewed in a direction perpendicular to and toward the back face of the subassemblies, a shortest distance between the subassemblies is at 0.1 cm and no more than 5.00 cm.

Embodiment 17A. The transducer apparatus of any one of Embodiment 1, 15 or 16 wherein the first electrode subassembly is positioned over a first target region in or on the subject's body and the second electrode subassembly is positioned over a second target region in or on the subject's body.

Embodiment 17B. The transducer apparatus of Embodiment 17A, wherein the first target region in or on the subject's body is a first tumor and the second target region in or on the subject's body is a second tumor different to the first tumor.

Embodiment 17C. The transducer apparatus of Embodiment 17A, wherein the first target region in or on the subject's body is a tumor and the second target region in or on the subject's body is a sentinel lymph node.

Embodiment 17D. The transducer apparatus of Embodiment 17A, wherein the first target region in or on the subject's body is a tumor in or on the subject's breast and the second target region in or on the subject's body is a sentinel lymph node in or around the subject's armpit.

Embodiment 17E. The transducer apparatus of Embodiment 1, wherein each subassembly further comprises a foam layer coupled to the front face of the anisotropic material layer or coupled to one or more portions thereof.

Embodiment 17F. The transducer apparatus of Embodiment 17E, wherein when viewed in a direction perpendicular to and toward the front face of the anisotropic material layer of each subassembly, the foam layer is or comprises a perimeter portion covering a perimeter of the front face of each subassembly.

Embodiment 17F-1. The transducer apparatus of Embodiment 17E, wherein when viewed in a direction perpendicular to and toward the front face of the anisotropic material layer of each subassembly, the foam layer is or comprises a perimeter portion covering a perimeter of each subassembly and extending around the front face of the perimeter of the subassembly to the back face of the perimeter of the subassembly thereby sealing an edge of each subassembly.

Embodiment 17G. The transducer apparatus of Embodiment 17E, wherein when viewed in a direction perpendicular to and toward the front face of the anisotropic material layer of each subassembly, the foam layer is or comprises a perimeter portion covering a perimeter of the anisotropic material layer.

Embodiment 17G-1. The transducer apparatus of Embodiment 17E, wherein when viewed in a direction perpendicular to and toward the front face of the anisotropic material layer of each subassembly, the foam layer is or comprises a perimeter portion covering a perimeter of the anisotropic material layer and extending around the front face of the perimeter of the anisotropic material layer to the back face of the perimeter of the anisotropic material layer thereby sealing an edge of each subassembly.

Embodiment 17H. The transducer apparatus of Embodiment 17E, wherein when viewed in a direction perpendicular to and toward the front face of the anisotropic material layer of each subassembly, a perimeter of the foam layer is larger than a perimeter of the anisotropic material layer.

Embodiment 17I. The transducer apparatus of Embodiment 17E, wherein when viewed in a direction perpendicular to and toward the front face of the anisotropic material layer of each subassembly, a shape of the perimeter of the foam layer is the same as or substantially the same as a shape of the perimeter of the anisotropic material layer.

Embodiment 17J. The transducer apparatus of Embodiment 1, further comprising at least one layer of conductive adhesive material or hydrogel located on a front facing side of the anisotropic material layer.

Embodiment 17K. The transducer apparatus of Embodiment 1, further comprising at least one layer of conductive adhesive material or hydrogel located on a back facing side of the anisotropic material layer.

Embodiment 17L. The transducer apparatus of Embodiment 1, wherein the first electrode subassembly is positioned over a first target region in or on the subject's body and the second electrode subassembly is positioned over a second target region in or on the subject's body, wherein either: (i) the first target region in or on the subject's body is a first tumor and the second target region in or on the subject's body is a second tumor different to the first tumor; or (ii) the first target region in or on the subject's body is a tumor and the second target region in or on the subject's body is a sentinel lymph node.

Embodiment 17M. The transducer apparatus of Embodiment 17L, wherein the first target region in or on the subject's body is a tumor in or on the subject's breast and the second target region in or on the subject's body is a sentinel lymph node in or around the subject's armpit.

Embodiment 18. The transducer apparatus of Embodiment 1, wherein each subassembly further comprises one or more printed circuit board connectors electrically connecting the at least one electrode element and the flexible electrical connector.

Embodiment 18A. The transducer apparatus of Embodiment 1, wherein each subassembly has at least two electrode elements.

Embodiment 18B. The transducer apparatus of Embodiment 1, wherein each subassembly has only one electrode element.

Embodiment 18C. The transducer apparatus of Embodiment 1, wherein each subassembly has one or more temperature sensors associated with some or all electrode elements of the at least one electrode element of each subassembly.

Embodiment 19. The transducer apparatus of Embodiment 1, wherein the anisotropic material layer of each subassembly has a different thermal and/or electrical conductivity in a direction perpendicular to the front face of the anisotropic material layer than in directions that are parallel to the front face of the anisotropic material layer.

Embodiment 20. The transducer apparatus of Embodiment 1, wherein the anisotropic material layer of each subassembly comprises graphite.

Embodiment 20A. The transducer apparatus of Embodiment 1, wherein the anisotropic material layer of each subassembly comprises a sheet of pyrolytic graphite, graphite foil made from compressed high purity exfoliated mineral graphite, or graphitized polymer film.

Embodiment 20B. The transducer apparatus of Embodiment 1, wherein when viewed in a direction perpendicular to and toward the back face of the subassemblies, each subassembly has a same shape.

Embodiment 20C. The transducer apparatus of Embodiment 20B, wherein when viewed in a direction perpendicular to and toward the back face of the subassemblies, each subassembly is triangular shaped, substantially triangular shaped, rounded triangular shaped, substantially rounded triangular shaped, kidney bean or jelly bean shaped, substantially kidney bean or jelly bean shaped, ovoid shaped, substantially ovoid shaped, oval shaped, or substantially oval shaped, or each subassembly has an asymmetric oval, ovaloid, ovoid, or ovate shape or a stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature.

Embodiment 20D. The transducer apparatus of Embodiment 1, wherein when viewed in a direction perpendicular to and toward the back face of the subassemblies, each electrode element has a same shape.

Embodiment 20E. The transducer apparatus of Embodiment 20D, wherein when viewed in a direction perpendicular to and toward the back face of the subassemblies, each electrode element is triangular shaped, substantially triangular shaped, rounded triangular shaped, substantially rounded triangular shaped, kidney bean or jelly bean shaped, substantially kidney bean or jelly bean shaped, ovoid shaped, substantially ovoid shaped, oval shaped, or substantially oval shaped, or each subassembly has an asymmetric oval, ovaloid, ovoid, or ovate shape or a stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature.

Embodiment 21. A transducer apparatus for delivering tumor treating fields to a subject's body, the transducer apparatus comprising: first and second electrode subassemblies, each subassembly configured to be positioned over the subject's body with a front face of the subassembly facing the subject's body, each subassembly having a back face opposite the front face, each subassembly comprising: at least one electrode element; and a flexible electrical connector electrically connecting the first and second electrode subassemblies, wherein the first and second electrode subassemblies are separated by the flexible electrical connector.

Embodiment 21A. The transducer apparatus of Embodiment 21, wherein the first and second electrode subassemblies are positioned over the subject's body on either side of the sternum.

Embodiment 22. The transducer apparatus of Embodiment 21, wherein the first electrode subassembly is positioned over a first target region in or on the subject's body and the second electrode subassembly is positioned over a second target region in or on the subject's body.

Embodiment 23. The transducer apparatus of Embodiment 22, wherein the first target region in or on the subject's body is a first tumor and the second target region in or on the subject's body is a second tumor different to the first tumor.

Embodiment 24. The transducer apparatus of Embodiment 22, wherein the first target region in or on the subject's body is a tumor and the second target region in or on the subject's body is a sentinel lymph node.

Embodiment 25. The transducer apparatus of Embodiment 24, wherein the first target region in or on the subject's body is a tumor in or on the subject's breast and the second target region in or on the subject's body is a sentinel lymph node in or around the subject's armpit.

Embodiment 26. A method of applying TTFields to treat one or more tumor or cluster of cancer cells in or on a subject's body, the method comprising: positioning a first transducer apparatus in a first initial position at a first location of the subject's body; positioning a second transducer apparatus in a second initial position at a second location of the subject's body; optionally positioning one or more other transducer apparatuses on the subject's body; inducing an electric field between the first transducer located at the first location of the subject's body and the second transducer located at the second location of the subject's body; wherein at least one transducer apparatus comprises: first and second electrode subassemblies, each subassembly configured to be positioned over the subject's body with a front face of the subassembly facing the subject's body, each subassembly having a back face opposite the front face, each subassembly comprising: at least one electrode element; and a flexible electrical connector electrically connecting the first and second electrode subassemblies, wherein the first and second electrode subassemblies are separated by the flexible electrical connector.

Embodiment 27. The method of Embodiment 26 wherein each of the first and second electrode subassemblies of the at least one transducer apparatus further comprises an anisotropic material layer electrically coupled to the at least one electrode element, the anisotropic material layer comprising a front face facing the subject's body and a back face opposite the front face, the back face facing the at least one first electrode element.

Embodiment 27A. The method of Embodiment 26 or 27, wherein the first and second electrode subassemblies are positioned on either side of the sternum.

Embodiment 27B. The method of Embodiment 26 or 27, wherein the first electrode subassembly is positioned over a first target region in or on the subject's body and the second electrode subassembly is positioned over a second target region in or on the subject's body, wherein either: (i) the first target region in or on the subject's body is a first tumor and the second target region in or on the subject's body is a second tumor different to the first tumor; or (ii) the first target region in or on the subject's body is a tumor and the second target region in or on the subject's body is a sentinel lymph node.

Embodiment 27C: The method of Embodiment 27B, wherein the first target region in or on the subject's body is a tumor in or on the subject's breast and the second target region in or on the subject's body is a sentinel lymph node in or around the subject's armpit.

Embodiment 28. The method of Embodiment 26 or 27 wherein the first electrode subassembly is positioned over a first target region in or on the subject's body and the second electrode subassembly is positioned over a second target region in or on the subject's body.

Embodiment 29. The method of Embodiment 28, wherein the first target region in or on the subject's body is a first tumor and the second target region in or on the subject's body is a second tumor different to the first tumor.

Embodiment 30. The method of Embodiment 28, wherein the first target region in or on the subject's body is a tumor and the second target region in or on the subject's body is a sentinel lymph node.

Embodiment 31. The method of Embodiment 30, wherein the first target region in or on the subject's body is a tumor in or on the subject's breast and the second target region in or on the subject's body is a sentinel lymph node in or around the subject's armpit.

Embodiment 32. The method of Embodiment 31, wherein both the first transducer apparatus and the second transducer apparatus comprise the first and second electrode subassemblies, wherein the first and second electrode subassemblies are separated by the flexible electrical connector.

Embodiment 33. The method of Embodiment 31, wherein the first transducer apparatus comprises the first and second electrode subassemblies, and the second transducer apparatus comprises a single electrode subassembly.

Embodiment 34. The method of any one of Embodiments 26-33, wherein at least one transducer apparatus comprises a transducer apparatus described herein.

Embodiment 35. A method of applying TTFields to treat one or more tumor or cluster of cancer cells in or on a subject's body, the method comprising: positioning a first transducer apparatus in a first initial position at a first location of the subject's body; positioning a second transducer apparatus in a second initial position at a second location of the subject's body; optionally positioning one or more other transducer apparatuses on the subject's body; inducing an electric field between the first transducer apparatus located at the first location of the subject's body and the second transducer apparatus located at the second location of the subject's body; wherein the first transducer apparatus is positioned over a first target region in or on the subject's body and the second transducer apparatus is positioned over a second target region in or on the subject's body, wherein the first target region is a tumor and the second target region is a sentinel lymph node.

Embodiment 36. The method of Embodiment 35 wherein at least one transducer apparatus further comprises an anisotropic material layer electrically coupled to at least one electrode element, the anisotropic material layer comprising a front face facing the subject's body and a back face opposite the front face, the back face facing the at least one electrode element.

Embodiment 36A. The method of claim 35 or 36, wherein the first and second electrode subassemblies are positioned over the subject's body on either side of the sternum.

Embodiment 37. The method of Embodiment 35 or 36, wherein the first target region in or on the subject's body is a tumor in or on the subject's breast and the second target region in or on the subject's body is a sentinel lymph node in or around the subject's armpit.

Embodiment 38. The method of Embodiment 35 or 36, wherein both the first transducer apparatus and the second transducer apparatus comprise first and second electrode subassemblies, wherein the first and second electrode subassemblies are separated by a flexible electrical connector.

Embodiment 39. The method of Embodiment 35 or 36, wherein the first transducer apparatus comprises first and second electrode subassemblies, wherein the first and second electrode subassemblies are separated by a flexible electrical connector, and wherein the second transducer apparatus comprises a single electrode subassembly.

Embodiment 40. The method of any one of Embodiments 35-39, wherein at least one transducer apparatus comprises a transducer apparatus described herein.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A transducer apparatus for delivering tumor treating fields to a subject's body, the transducer apparatus comprising:
first and second electrode subassemblies, each subassembly configured to be positioned over the subject's body with a front face of the subassembly facing the subject's body, each subassembly having a back face opposite the front face, each subassembly comprising:
at least one electrode element;
an anisotropic material layer electrically coupled to the at least one electrode element, the anisotropic material layer comprising a front face facing the subject's body and a back face opposite the front face, the back face facing the at least one first electrode element; and
a flexible electrical connector electrically connecting the first and second electrode subassemblies,
wherein the first and second electrode subassemblies are separated by the flexible electrical connector.

2. The transducer apparatus of claim 1, wherein each subassembly further comprises a substrate for holding the at least one electrode element and the anisotropic material layer against the subject's body, wherein the substrate of each subassembly comprises a front face facing the subject's body and a back face opposite the front face, the front face of the substrate facing the at least one first electrode element and the anisotropic material layer, and wherein the substrates of the first and second electrode subassemblies are separated by the flexible electrical connector.

3. The transducer apparatus of claim 2, wherein the substrates of the first and second electrode subassemblies are distinct and not a unitary body.

4. The transducer apparatus of claim 1, wherein the flexible electrical connector electrically connects the at least one electrode element of the first electrode subassembly and the at least one electrode element of the second electrode subassembly.

5. The transducer apparatus of claim 1, wherein when viewed in a direction perpendicular to and toward the back face of the subassemblies, the flexible electrical connector has a linear shape.

6. The transducer apparatus of claim 1, wherein when viewed in a direction perpendicular to and toward the back face of the subassemblies, the flexible electrical connector has a U-shape, a substantially U-shape, or a rounded V-shape.

7. The transducer apparatus of claim 1, wherein the flexible electrical connector is adapted to be deformed to a substantially concave shape away from the subject's body.

8. The transducer apparatus of claim 1, wherein when the subassemblies are viewed in cross-section, the flexible electrical connector is adapted to be deformed to have a void between the flexible electrical connector and the subject's body.

9. The transducer apparatus of claim 1, wherein one or more wedges reside between the flexible electrical connector and the subject's body, optionally wherein the one or more wedges are comprised of foam material.

10. The transducer apparatus of claim 1, wherein the flexible electrical connector comprises a front face facing the subject's body and a back face opposite the front face, wherein the front face of the flexible electrical connector is free of adhesive material.

11. The transducer apparatus of claim 1, wherein the flexible electrical connector comprises a front face facing the subject's body and a back face,
wherein the transducer apparatus further comprises a foam layer on at least one of the front face or the back face of the flexible electrical connector.

12. The transducer apparatus of claim 1, wherein the flexible electrical connector comprises a front face facing the subject's body and a back face,
wherein the transducer apparatus further comprises an anisotropic material layer on the front face of the flexible electrical connector.

13. The transducer apparatus of claim 12, wherein the anisotropic material layer on the front face of the flexible electrical connector forms a unitary body with respective anisotropic material layers of the subassemblies.

14. The transducer apparatus of claim 1, wherein the anisotropic material layer of each subassembly comprises graphite.

15. The transducer apparatus of claim 1, wherein the first electrode subassembly is positioned over a first target region in or on the subject's body and the second electrode subassembly is positioned over a second target region in or on the subject's body, wherein either:
(i) the first target region in or on the subject's body is a first tumor and the second target region in or on the subject's body is a second tumor different to the first tumor; or
(ii) the first target region in or on the subject's body is a tumor and the second target region in or on the subject's body is a sentinel lymph node.

16. The transducer apparatus of claim 15, wherein the first target region in or on the subject's body is a tumor in or on the subject's breast and the second target region in or on the subject's body is a sentinel lymph node in or around the subject's armpit.

17. A method of applying TTFields to treat one or more tumor or cluster of cancer cells in or on a subject's body, the method comprising:
positioning a first transducer apparatus in a first initial position at a first location of the subject's body;
positioning a second transducer apparatus in a second initial position at a second location of the subject's body;
optionally positioning one or more other transducer apparatuses on the subject's body;
inducing an electric field between the first transducer located at the first location of the subject's body and the second transducer located at the second location of the subject's body; wherein:
at least one transducer apparatus comprises:
first and second electrode subassemblies, each subassembly configured to be positioned over the subject's body with a front face of the subassembly facing the subject's body, each subassembly having a back face opposite the front face, each subassembly comprising: at least one electrode element;
an anisotropic material layer electrically coupled to the at least one electrode element, the anisotropic material layer comprising a front face facing the subject's body and a back face opposite the front face, the back face facing the at least one first electrode element; and
a flexible electrical connector electrically connecting the first and second electrode subassemblies, wherein the first and second electrode subassemblies are separated by the flexible electrical connector.

18. The method of claim 17, wherein the first and second electrode subassemblies are positioned over the subject's body on either side of the sternum.

19. The method of claim 17, wherein the first electrode subassembly is positioned over a first target region in or on the subject's body and the second electrode subassembly is positioned over a second target region in or on the subject's body, wherein either:
(i) the first target region in or on the subject's body is a first tumor and the second target region in or on the subject's body is a second tumor different to the first tumor; or
(ii) the first target region in or on the subject's body is a tumor and the second target region in or on the subject's body is a sentinel lymph node.

20. The method of claim 19, wherein the first target region in or on the subject's body is a tumor in or on the subject's breast and the second target region in or on the subject's body is a sentinel lymph node in or around the subject's armpit.
